(19) United States
(12) Patent Application Publication
(10) Pub. No.: US 2026/0284281 A1
(43) Pub. Date: Sep. 24, 2026

(54) WEARABLE HOT-COMPRESS BREAST PUMP AND METHOD FOR CONTROLLING HOT-COMPRESS BREAST PUMPS

(71) Applicant: SHENZHEN TPH TECHNOLOGY CO., LTD., Guangdong (CN)

(72) Inventor: Xianzi CHEN

(21) Appl. No.: 19/491,918

(22) PCT Filed: Mar. 31, 2023
(86) PCT No.: PCT/CN2023/085683

§ 371 Date: Jun. 15, 2026

(30) Foreign Application Priority Data
Nov. 18, 2022 (CN) 202211448893.7

(51) Int. Cl. A61M 1/06; A61F 7/00; A61F 7/02

(52) U.S. Cl. CPC A61M 1/062; A61F 7/007; A61F 2007/0021; A61F 2007/0093; A61F 2007/0096; A61F 2007/0239; A61F 2007/0257

(57) ABSTRACT

A wearable hot-compress breast pump and a method for controlling the breast pump, which includes a milk suctioning piece (1), which has one end serving as a milk outlet tube (11) and the other end serving as a funnel-shaped cover (12) communicating with the milk outlet tube (11), and an outer diameter of which gradually increases in a direction away from the milk outlet tube (11); a principal unit (2), having a casing (21), on one end of which a milk-storing chamber (211) is disposed, and on the other end of which a funnel-shaped chamber (212) that matches the shape of the funnel-shaped cover (12) is disposed; a milk inlet tube (213) communicating with funnel-shaped chamber (212), which is disposed inside milk-storing chamber (211), wherein the milk suctioning piece (1) is stuck into the interior of the inlet tube (213) through the outlet tube (11), so as to connect the principal unit (2); a heating apparatus, which is disposed one side of the funnel-shaped cover (12) facing the outlet tube (11) and connected with the funnel-shaped cover (12); a power supply device and a controller, which are arranged inside the casing (21) and electrically connected to the heating apparatus; and a temperature sensor (6), which is embedded inside the funnel-shaped cover (12) and electrically connected to the power supply device. The wearable hot-compress breast pump can be worn inside a bra for convenient and easy use; the funnel-shaped cover (12) contains a heating element (3) to achieve a hot-compress and massage effect; in addition, it is possible to set and control heating rates, temperature, and battery life through material selection and calculations.

### FIELD OF THE INVENTION

The invention relates to the technical field of breast pumps, in particular to a hot-compress breast pump especially suitable for wear in underwear and a method for controlling the hot-compress breast pump.

### BACKGROUND OF THE INVENTION

Mothers who are breastfeeding often apply a hot compress on their breasts during nursing by using a hot towel or a heatable massager, so as to improve secretion efficiency of milk, and then use a breast pump to suction milk for nourishing their babies. This method of applying a hot compress for suctioning milk increases the number of apparatuses and costs necessary for an entire process of applying a hot compress for suctioning milk and is poor in portability; in addition, it also takes a certain amount of time to suction breast milk. Therefore, in a process of using a breast pump to suction breast milk, a breast is enclosed by the breast pump; as a result, it is impossible to adopt the above-mentioned method and use a heatable massager to apply a hot compress during such a process; instead, mothers have no choice but to apply a hot compress on their breasts before suctioning milk; thus they have to stop applying a hot compress in a subsequent process of suctioning milk; consequently, this method of applying a hot compress on breasts before suctioning milk has very limited effectiveness.

Currently, some breast pumps available on the market have a function of simultaneously applying a hot compress and suctioning milk, but these breast pumps are of a manually-handled type and need to be held steady during use; in addition, connections and hoses between a milk bottle and a breast pump causes inconvenience in use and portability. In addition, this type of breast pumps has low heating efficiency and usually performs heating at a fixed rate, making it impossible to achieve an experience of applying a hot compress and suctioning milk in a customized manner. Therefore, how to invent a wearable breast pump capable of applying a hot compress while suctioning milk is a technical problem that urgently needs to be solved in this field.

### SUMMARY OF THE INVENTION

In order to solve the technical problem of absence of a wearable breast pump capable of applying a hot compress while suctioning milk in the prior art, the present invention provides a wearable hot-compress breast pump especially suitable for wear in underwear.

In order to solve the above technical problem, the technical scheme adopted in the present invention is as follows:
the present invention provides a wearable hot-compress breast pump, which comprises:
a milk suctioning piece, which has one end serving as a milk outlet tube and the other end serving as a funnel-shaped cover, which communicates with the milk outlet tube, and an outer diameter of which gradually increases in a direction away from the milk outlet tube;
a principal unit, which has a casing, on one end of which a milk-storing chamber is disposed, and on the other end of which a funnel-shaped chamber that matches the shape of the funnel-shaped cover is disposed; wherein the milk-storing chamber has a milk inlet tube communicating with funnel-shaped chamber;
and the milk suctioning piece is stuck into the interior of the inlet tube through the outlet tube, so as to connect the principal unit;
a heating apparatus, which is disposed on one side of the funnel-shaped cover facing the milk outlet tube and connected with the funnel-shaped cover;
a power supply device and a controller, which are arranged inside the casing and electrically connected to the heating apparatus; and
a temperature sensor, which is embedded inside the funnel-shaped cover and electrically connected to the power supply device.

Furthermore, the heating apparatus includes at least one heating element, which is embedded in a side surface of the funnel-shaped cover facing the milk outlet tube and the heating element extends around a surface contour of the funnel-shaped cover in a spiral manner.

Furthermore, the heating apparatus also includes a heating cover, which is in the shape of a funnel that matches the shape of the funnel-shaped cover, and the heating element is arranged on one side of the heating cover facing the funnel-shaped cover, and the heating cover is provided with a mounting hole that matches the shape of the milk outlet tube, and the heating cover encases an outside of the milk outlet tube through the mounting hole and fits onto one side of the funnel-shaped cover facing the milk outlet tube, and encloses the heating element between the heating cover and the funnel-shaped cover.

Preferably, four contacts are set on one side of the funnel-shaped cover facing the milk outlet tube, and correspondingly connected to positive and negative terminals of the heating element and positive and negative terminals of the temperature sensor respectively;

and the other end of the casing is provided with four contacts electrically connected to the power supply device, which are correspondingly connected to the four contacts set on the funnel-shaped cover respectively.

Preferably, the four contacts of the funnel-shaped cover and the four contacts of the casing are correspondingly connected with each other as a whole by means of a waterproof coating, and a magnetic connection is formed through pair magnets between the four contacts of the funnel-shaped cover and the four contacts of the casing, which have been connected with each other as a whole.

Furthermore, the heating apparatus includes:
an induction coil mounted on the heating cover, and the induction coil being electrically connected to the power supply device by joining the contacts set on the heating cover and the contacts set on the casing together, and
a metal induction heating element embedded inside the funnel-shaped cover.

Preferably, the heating element is a resistive heating sheet, a silicone heating sheet, a mica heating sheet, a MCH heating sheet, a ceramic heating sheet, a silicon-conductive heating sheet, a flexible carbon film underfloor heating sheet, a PET electrothermal film polyester heating sheet, an aluminum foil heating sheet, a cushion heating sheet, a new-type surface heating sheet or a new-type thin-film heating sheet. The heating element is in the form of strips, sheets, wires, or planes.

A method for controlling the wearable hot-compress breast pump provided by the present invention includes the steps of:
inputting a corresponding hot-compress control command into a control interface of a principal unit on demands;
enabling a controller to receive the hot-compress control command and generate a corresponding current-controlling command;
enabling a power supply device to heat a heating apparatus in a corresponding heating mode according to the current-controlling command;
enabling a temperature sensor to sense a temperature of the heating apparatus and feed the temperature back to the controller; and
enabling the controller to judge whether the temperature of the heating apparatus has reached a preset threshold of the corresponding heating mode, if yes, controlling the power supply device to stop heating the heating apparatus; if not, controlling the power supply device to continue to heat the heating apparatus in a corresponding heating mode until the temperature of the heating apparatus reaches the preset threshold of the corresponding heating mode, then making the wearable hot-compress breast pump enter a heat retention mode.

Preferably, the step of enabling a power supply device to heat a heating apparatus in a corresponding heating mode according to the current-controlling command, includes:

the sub-step of enabling the controller to regulate heating power of a heating element by matching a PWM waveform corresponding to the heating mode, so as to control a rate at which a funnel-shaped cover reaches a preset temperature.

Preferably, the step of enabling a power supply device to heat a heating apparatus in a corresponding heating mode according to the current-controlling command, includes:
the sub-steps of enabling the controller to control a current alternating fluctuation in a circuit inside a coil holder by matching a PWM waveform corresponding to the heating mode, then enabling an induction coil to generate an alternating induced magnetic field synchronous with the current alternating fluctuation, next enabling a metal induction heating element to synchronously produce eddy currents and heat in the alternating induced magnetic field, so as to control a rate at which a funnel-shaped cover reaches a preset temperature.

Compared with the prior art, the wearable hot-compress breast pump provided by the present invention has the following advantages:
during using the wearable hot-compress breast pump, it is unnecessary to manually hold it, and it can be worn inside a bra for on-the-go use, and by way of integrating a breast pump and a hot-compress massager, it is possible to raise convenience and reduce costs;
the design of embedding the heating element inside the funnel-shaped cover, compared to a conventional design of arranging it outside the funnel-shaped cover, performs a hot-massage function without affecting the overall appearance of the breast pump; in addition, the funnel-shaped cover with a built-in heating element is a detachable component, which has advantages of low-cost maintenance, convenience, and easiness to clean;
the contacts between the funnel-shaped cover and the casing of the principal unit employ a waterproof magnetic contact structure, preventing short circuits and damage caused by water ingress, and improving reliability and safety of a breast pump product;
the funnel-shaped cover has a built-in temperature sensor that can accurately detect a temperature of the funnel-shaped cover in real time and provide real-time feedback of the detected temperature to a control system, enabling precise regulation and control to temperatures of the funnel-shaped cover;
the casing of the principal unit is provided with a switch for controlling hot-compress massage; therefore, it is possible for users to conveniently choose whether to use the hot-compress massage function based on ambient temperatures;
the casing of the principal unit has multiple temperature settings for the hot-compress massage function; therefore, it is possible for users to choose different comfortable hot-compress temperatures based on their personal experience;
the heating element embedded inside the funnel-shaped cover can be precisely controlled through the controller based on different PWM waveforms, so as to adjust a rate at which temperatures of the funnel-shaped cover rise, preventing local overheating and enabling the funnel-shaped cover to be heated evenly;
the hot-compress massage function can simulate an oral temperature at which a baby is suckling, stimulate and decongest a mammary gland, and increase milk production and breastfeeding efficiency; in particular, it can enhance users' experience and comfort in cold weather;
in order to meet users' needs for a hot compress, it is possible to make a reasonable design in heating rate, temperature, and battery life by way of selecting materials and performing calculations.

### BRIEF DESCRIPTION OF THE DRAWINGS

In order to more clearly describe the technical solution in an embodiment provided by the present invention, we shall make a description to figures necessary for description in the embodiment and the prior art as follows. It is obvious that the figures described herein are merely for some embodiments of the present invention. For a person skilled in the art, it is possible to obtain other figures based on these figures without inventive work.

FIG. 1 is a perspective view showing assemble of the wearable hot-compress breast pump in Embodiment 1 according to the present invention.

FIG. 2 is a front view showing assemble of the wearable hot-compress breast pump in Embodiment 1 according to the present invention.

FIG. 3 is a back view showing assemble of the wearable hot-compress breast pump in Embodiment 1 according to the present invention.

FIG. 4 is a side view showing assemble of the wearable hot-compress breast pump in Embodiment 1 according to the present invention.

FIG. 5 is a top view showing assemble of the wearable hot-compress breast pump in Embodiment 1 according to the present invention.

FIG. 6 is an exploded perspective view (1) of the wearable hot-compress breast pump in Embodiment 1 according to the present invention.

FIG. 7 is an exploded perspective view (2) of the wearable hot-compress breast pump in Embodiment 1 according to the present invention . . .

FIG. 8 is a curve graph showing how the MCU of the wearable hot-compress breast pump of the present invention controls the funnel-shaped cover to raise temperatures through different PWM waveforms . . .

FIG. 9 is a control flowchart of the heating sheet used in the heating apparatus of the method for controlling the wearable hot-compress breast pump of the present invention.

FIG. 10 is a control flowchart of the induction coil used in the heating apparatus of the method for controlling the wearable hot-compress breast pump of the present invention.

Where, 1—milk suctioning piece; 11—milk outlet tube; 12—funnel-shaped cover; 2—principal unit; 21—casing; 211—milk-storing chamber; 212—funnel-shaped chamber; 213—milk inlet tube; 3—heating element; 4—rigid contact; 5—elastic contact; 6—temperature sensor.

### DETAILED DESCRIPTION OF SOME EMBODIMENTS

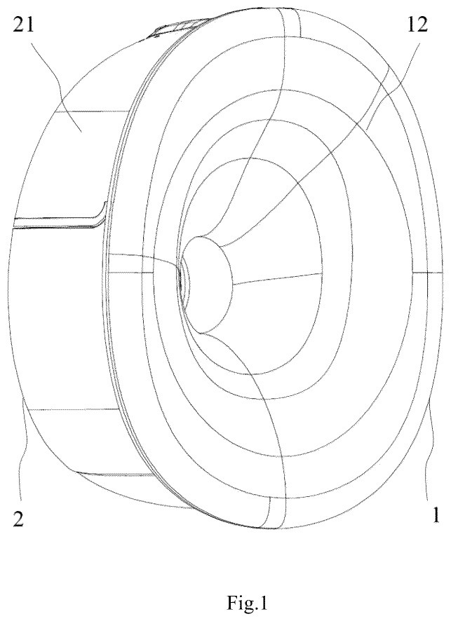

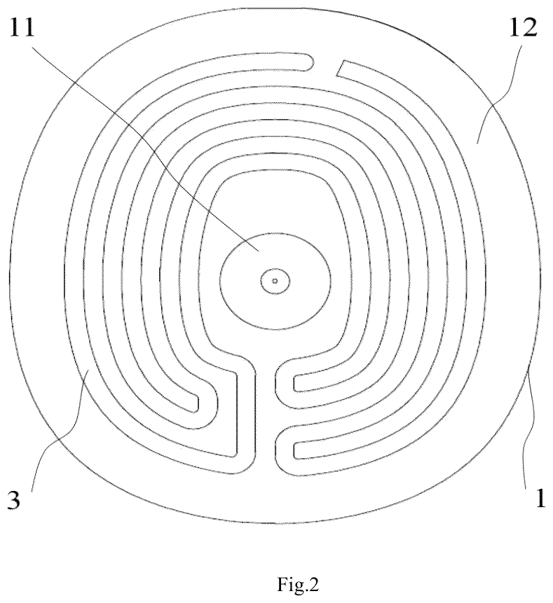

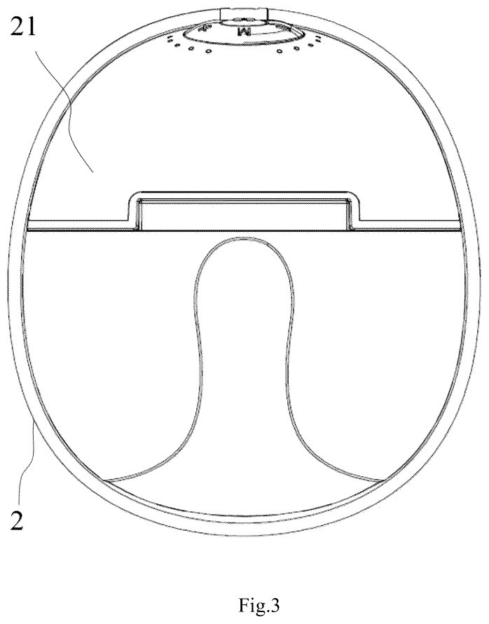

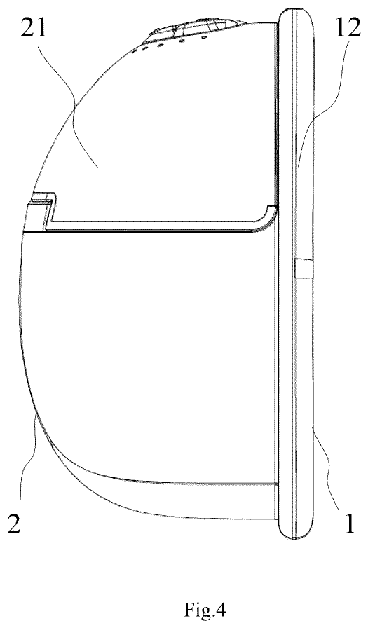

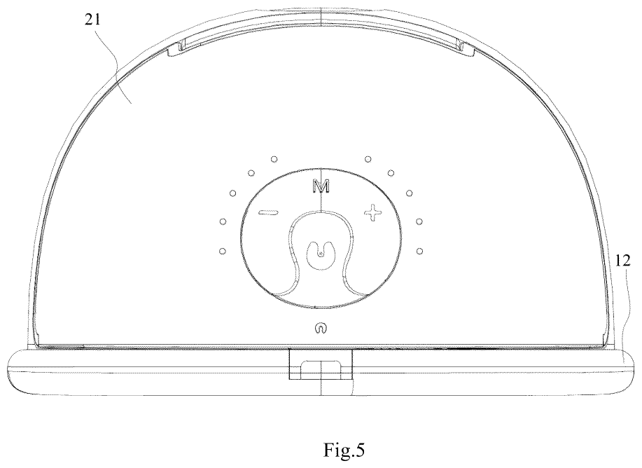

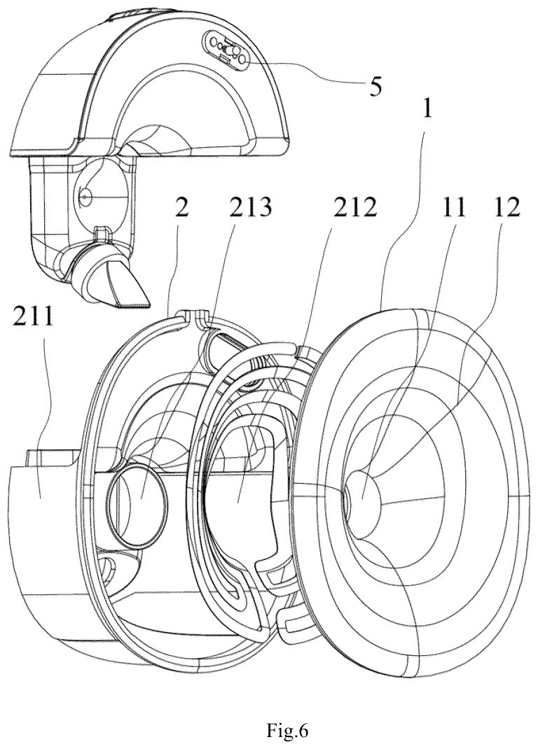

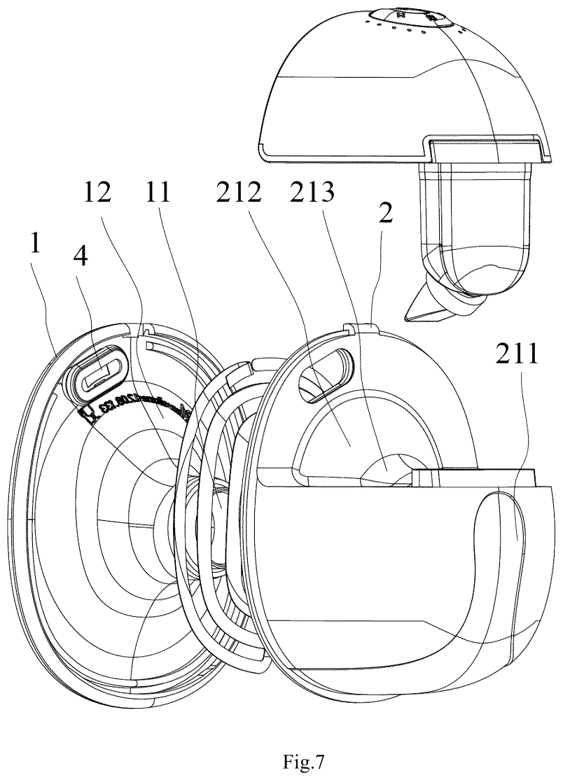

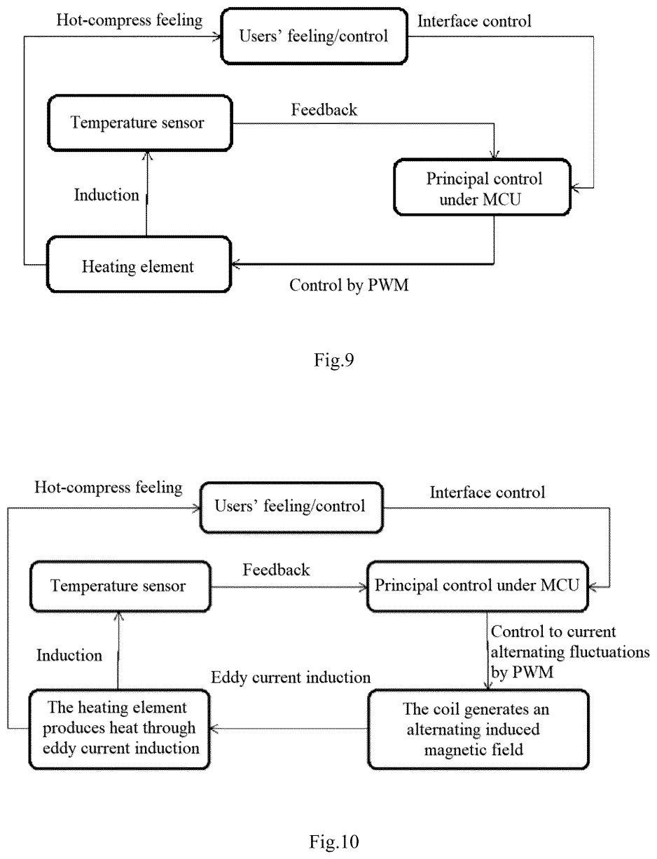

In order to make the technical problem to be solved by the present invention, the technical solution, and the beneficial effects clearer, we shall further describe in detail the principle and structure of the present invention with reference to FIGS. 1-10 and examples. It should be understood that the examples described herein are only used to explain the present invention and not pose any limitations on the present invention.

In the technical field of breast pumps, a Chinese patent (CN101340937A) discloses a handheld breast pump with an electrically-heating cover, which includes a breast cover that performs a function of electric heating and is made of two-component or multi-component materials, and a heating element that is made from plastic material where conductive carbon fiber is added. It has a complex structure and high process requirements, and control to temperatures of the heating element outside a funnel opening is mainly performed through an external temperature sensor. The properties of carbon fiber cause low heating efficiency and difficulty in control to temperature, making it difficult to achieve an ideal hot-compress effect. In addition, this breast cover is used in the handheld breast pump, so it is necessary to arrange a hose and connection between a milk bottle and a breast pump, causing inconvenience in use and portability.

A Chinese patent (CN215134379U) discloses a heatable breast pump with a far-infrared electrically-heating film. The far-infrared electrically-heating film is either directly printed on a breast cover or printed on a frame, which is then assembled onto a breast cover to heat the breast cover. Since the far-infrared electrically-heating film is directly printed on the breast cover, in cases of applying too much force on the breast cover or rubbing the breast cover during washing, the far-infrared electrically-heating film may easily be damaged. If the far-infrared electrically-heating film is printed on an inner surface of the breast cover, the far-infrared electrically-heating film will come into direct contact with and heat breast milk; in this way, it easily damages milk's components or even contaminate it. If the far-infrared electrically-heating film is printed on an outer surface of the breast cover, the far-infrared electrically-heating film will come into direct contact with the breast; in this way, it may cause local overheating and scald the breast. If the far-infrared electrically-heating film is printed on a frame, which is then installed into the breast cover for heating, it is possible to affect heat conduction and reduce heating efficiency due to gaps at the contacts during assembly, thereby affecting hot-compress temperature and its effectiveness. In addition, this breast pump is also a handheld breast pump, causing inconvenience in use and portability likewise.

With reference to FIGS. 1-7, a wearable hot-compress breast pump according to the present invention, which comprises:
a milk suctioning piece 1, a principal unit 2, a heating apparatus, a power supply device, a controller, and a temperature sensor; the milk suctioning piece 1 has one end serving as a milk outlet tube 11 and the other end serving as a funnel-shaped cover 12, which communicates with the milk outlet tube, and an outer diameter of which gradually increases in a direction away from the milk outlet tube;
the principal unit 2 has a casing 21, on one end of which a milk-storing chamber 211 is disposed, and on the other end of which a funnel-shaped chamber 212 that matches the shape of the funnel-shaped cover 12 is disposed; and the milk-storing chamber 211 has a milk inlet tube 213 communicating with the funnel-shaped chamber 212;
the milk suctioning piece 1 is stuck into the interior of the inlet tube 213 through the outlet tube 11, so as to connect the principal unit 2;
the heating apparatus is disposed on one side of the funnel-shaped cover 12 facing the milk outlet tube 11 and connected the funnel-shaped cover 12;
the power supply device and the controller are arranged inside the casing 21 of the principal unit 2 and electrically connected to the heating apparatus. Preferably, the controller is an MCU controller.

The temperature sensor 6 is embedded inside the funnel-shaped cover 12 and electrically connected to the power supply device, and the temperature sensor is configured to monitor a temperature of the funnel-shaped cover 12 in real time and feed it back to the controller to achieve precise control to the temperature of the funnel-shaped cover 12.

As Embodiment 1 of the present invention, the heating apparatus includes at least one heating element 3, which is embedded in a side surface of the funnel-shaped cover 12 facing the milk outlet tube 11 and the heating element 3 is strip-shaped and extends around a surface of the funnel-shaped cover 12 in a spiral manner, so that the funnel-shaped cover 12 is heated evenly, so as to enable a temperature both on a surface in contact with the breast and a surface in contact with the funnel shaped cover 12 back facing the milk outlet tube 11 to be uniform. In other examples, the heating element 3 may also have a sheet structure. In this embodiment, preferably the heating element 3 is embedded inside one side surface of the funnel-shaped cover 12 facing the milk outlet tube 11 by means of injection molding. The heating element 3 is any one of an USB heating piece 3, a silicone heating piece (silicone rubber heating piece 3), a mica heating piece 3, a MCH heating piece 3, a ceramic heating piece 3, a silicon-conductive heating piece 3, a flexible carbon film underfloor heating piece 3, a PET electrothermal film polyester heating piece 3, an aluminum foil heating piece 3, a cushion heating piece 3, a new-type surface heating piece 3, a new-type thin-film heating piece 3 and the like. The heating element 3 may be in the form of strips, sheets, wires, or planes.

As Embodiment 2 of this embodiment (not shown in the figures), the heating apparatus further includes a heating cover, which is in the shape of a funnel that matches the shape of the funnel-shaped cover 12. The heating element 3 is disposed on one side of the heating cover facing the funnel-shaped cover 12. The heating cover is provided with a mounting hole that matches the shape of the milk outlet tube 11, and the heating cover encases an outside of the milk outlet tube 11 through the mounting hole and fits onto one side of the funnel-shaped cover 12 facing the milk outlet tube 11, and encloses the heating element 3 between the heating cover and the funnel-shaped cover 12. In this example, preferably the heating cover is a funnel-shaped plastic piece, which can encase and clasp the outside of the milk outlet tube 11 through elastic deformation, or can be screwed to the outside of the milk outlet tube 11.

In this embodiment, four contacts are set on one side of the funnel-shaped cover 12 facing the milk outlet tube 11, and correspondingly connected to positive and negative terminals of the heating element 3 and positive and negative terminals of the temperature sensor 6 respectively. Four contacts electrically connected to the power supply device are set on the other end of the casing 21 of the principal unit 2, and after assembling the funnel-shaped cover 12 correspondingly with the casing 21, are electrically connect with the four contacts set on the funnel-shaped cover 12. As an example, the four contacts set on the funnel-shaped cover 12 are a rigid contact 4, and the four contacts set on the casing 21 are an elastic contact 5. In another example, the four contacts set on the funnel-shaped cover 12 are an elastic contact 5, and the four contacts set on the casing 21 are a rigid contact 4. In other examples, all the contacts set on the funnel-shaped cover 12 and the contacts set on the casing 21 are either an elastic contact 5 or a rigid contact 4.

As a preferred example of this embodiment, the four rigid contacts 4 and the four elastic contacts 5 are correspondingly connected with each other as a whole by means of a waterproof coating, so as avoid damage to the rigid contacts 4 and the elastic contacts 5 caused by a short circuit due to water ingress. As a preferred example of this embodiment, a magnetic connection is formed through pair magnets between the four rigid contacts 4 and the four elastic contacts 5, which have been connected with each other as a whole. After the casing 21 of the principal unit 2 and the funnel-shaped cover 12 are assembled with each other, the four rigid contacts 4 and the four elastic contacts 5 tightly join each other through magnetic attraction between the magnets.

A heat conduction temperature of the funnel-shaped cover 12 of the wearable hot-compress breast pump provided in this embodiment is calculated as follows.

According to formulas Q=cm(T2−T1) and W=Q/t, where Q represents an amount of heat absorbed or released by materials (unit: J); c represents a specific heat capacity of materials (unit: J/(g·k)); m represents mass of materials (unit: g); (T2−T1) represents a temperature difference of materials before and after heating or cooling (unit: ° C.); W represents work done by an energized heating element (unit: w); t represents time necessary to heat a funnel-shaped cover to a preset temperature (unit: s). By means of the parameters in the above formulas, it is possible to calculate required heating power, as well as battery specifications and design requirements of the power supply device, based on a desired preset temperature and heating time. For example, increasing a temperature of the funnel-shaped cover 12 from T1=25° C. to T2=45° C., then finding out a specific heat capacity c of the material used in the funnel-shaped cover 12, and weighing out the mass m of the funnel-shaped cover 12, then calculating an amount of heat Q absorbed by the funnel-shaped cover 12 from 25° C. to 45° C., based on the set heating time t=2 minutes for the funnel-shaped cover 12 to complete a temperature rise from 25° C. to 45° C.; in this way, it is possible to calculate required heating power W. Then, based on the heating power and required duration for once using a battery, it is possible to calculate a required lithium battery capacity. Tables 1-3 show data based on an assumption of heating the funnel-shaped cover 12 of the wearable hot-compress breast pump in this embodiment for 2 minutes, 1 minute, and 30 seconds, respectively. The tables list the design requirements for the hot-compress function based on three assumptions of setting heating time.

TABLE 1
DESIGN REQUIREMENTS FOR THE HOT-COMPRESS FUNCTION

| | |
|---|---|
| Assumption on how long to use the | 60-80 min (take a value of 70 min = 1.17 h) |
| breast pump | |
| Assumption on heating the funnel- | A rise from 22° C. to 40° C. |
| shaped cover to a preset temperature | |
| Assumption on how long to heat the | 2 min |
| funnel-shaped cover | |
| Electricity consumed when the | 3.7*0.5*1.17 min = 2.16 Wh |
| breast pump operates for 70 minutes | |
| (assuming that pump valve's current | |
| is 0.5 A) | |
| According to a calculation formula | formula 1: Q = cm(T2 − T1), where Q represents |
| | an amount of heat absorbed or released by materials |
| | (unit: J); c represents a specific heat capacity of |
| | materials (unit: J/(g · k)); m represents mass |
| | of materials (unit: g); (T2 − T1) represents a |
| | temperature difference of materials before and after |
| | heating or cooling (unit: ° C.). (get the specific |
| | heat capacity of silicone rubber by searching c = |
| | 1.27 J/g*° C.) |
| Substitute the data into formula 1 to | Q = 1.27 (J/g*° C.)*56 g*18° C. = 1280.16 J |
| calculate the amount of heat | |
| absorbed by the funnel-shaped cover | |
| from 22° C. to 40° C. | |
| According to a calculation formula | formula 2: W = Q/t, W represents work done by the |
| | energized heating element (unit: w); t represents time |
| | necessary to heat the funnel- shaped cover to a preset |
| | temperature (unit: s). |
| Substitute the data into formula 2; | W = 1280.16/120 = 10.67 W, Assuming a resistance |
| the work required for a heating wire | of 2.5Ω, the current would be 2A. |
| to release heat | |
| The heating wire does not work | During suctioning milk, the heating wire has total |
| continuously. Apart from heating to | power of 10.67 W |
| 40° C. in the first 2 minutes, it then | * 0.58 = 6.2 Wh; therefore, it is expected to use |
| stops heating. After it cools down to | a lithium battery of 3.7 V/1700 mAh = 6.29 Wh. |
| 38° C., try to heating again to 40° C. | |
| Assuming it takes 30 seconds to | |
| drop from 40° C. to 38° C. and 30 | |
| seconds to heat from 38° C. to 40° C., | |
| the total power-consuming heating | |
| time for the wire is 35 minutes, and | |
| in the remaining 35 minutes there is | |
| no consumption of electricity with | |
| heat retention. 35 min = 0.58 h | |
| If the heating efficiency is calculated | A battery of 6.29 + 6.29*(1-80%) = 7.55 Wh |
| at 80% | is actually required |
| The total required lithium battery | Because the breast pump operates for 70 minutes and |
| capacity is | requires 2.16 Wh, the total battery capacity needs |
| | to be at least 2.16 × 7.55 = 9.7 Wh = |
| | 3.7 V/2700 mAh. |

Note:
The above calculations give theoretical values, and there may be some deviations in practice.

TABLE 2
DESIGN REQUIREMENTS FOR THE HOT-COMPRESS FUNCTION

| | |
|---|---|
| Assumption on how long to use the | 60-80 min (take a value of 70 min = 1.17 h) |
| breast pump | |
| Assumption on heating the funnel- | A rise from 22° C. to 40° C. |
| shaped cover to a preset temperature | |
| Assumption on how long to heat the | 1 min |
| funnel-shaped cover | |
| Electricity consumed when the breast | 3.7*0.5*1.17 min = 2.16 Wh |
| pump operates for 70 minutes (assuming | |
| that pump valve's current is 0.5 A) | |
| According to a calculation formula | formula 1: Q = cm(T2 − T1), where Q |
| | represents an amount of heat absorbed or released |
| | by materials (unit: J); c represents a specific |
| | heat capacity of materials (unit: J/(g · k)); |
| | m represents mass of materials (unit: g); |
| | (T2 − T1) represents a temperature difference |
| | of materials before and after heating or cooling |
| | (unit: ° C.). (get the specific heat capacity of |
| | silicone rubber by searching c = 1.27 J/g*° C.) |
| Substitute the data into formula 1 to | Q = 1.27 (J/g*° C.)*56 g*18° C. = |
| calculate the amount of heat absorbed | 1280.16 J |
| by the funnel-shaped cover from 22° C. | |
| to 40° C. | |
| According to a calculation formula | formula 2: W = Q/t, W represents work done by the |
| | energized heating element (unit: w); t represents time |
| | necessary to heat the funnel-shaped cover to a preset |
| | temperature (unit: s). |
| Substitute the data into formula 2; | W = 1280.16/60 = 21.33 W |
| the work required for a heating wire | |
| to release heat | |
| The heating wire does not work | During suctioning milk, the heating wire has total power |
| continuously. Apart from heating to | of = 21.33 W*0.58 = 12.37 Wh; therefore, it is |
| 40° C. in the first 2 minutes, it then stops | expected to use a lithium battery of 3.7 V/3400 mAh = |
| heating. After it cools down to 38° C., try | 12.58 Wh. |
| to heating again to 40° C. Assuming it | |
| takes 30 seconds to drop from 40° C. to | |
| 38° C. and 30 seconds to heat from 38° C. | |
| to 40° C., the total power-consuming | |
| heating time for the wire is 35 minutes, | |
| and in the remaining 35 minutes there is | |
| no consumption of electricity with heat | |
| retention. 35 min = 0.58 h | |
| If the heating efficiency is calculated at | A battery of 12.58 + 12.58*(1-80%) = 15.1 Wh |
| 80% | is actually required |
| The total required lithium battery | Because the breast pump operates for 70 minutes and |
| capacity is | requires 2.16 Wh, the total battery capacity needs to |
| | be at least 2.16 + 15.1 = 17.3 Wh = |
| | 3.7 V/4700 mAh |

Note:
The above calculations give theoretical values, and there may be some deviations in practice.

TABLE 3
DESIGN REQUIREMENTS FOR THE HOT-COMPRESS FUNCTION

| | |
|---|---|
| Assumption on how long to use the | 60-80 min (take a value of 70 min = 1.17 h) |
| breast pump | |
| Assumption on heating the funnel- | A rise from 22° C. to 40° C. |
| shaped cover to a preset temperature | |
| Assumption on how long to heat the | 30 s |
| funnel-shaped cover | |
| Electricity consumed when the breast | 3.7*0.5*1.17 min = 2.16 Wh |
| pump operates for 70 minutes (assuming | |
| that pump valve's current is 0.5 A) | |
| According to a calculation formula | formula 1: Q = cm(T2 − T1), where Q represents |
| | an amount of heat absorbed or released by materials |
| | (unit: J); c represents a specific heat capacity of |
| | materials (unit: J/(g · k)); m represents mass of |
| | materials (unit: g); (T2 − T1) represents a temperature |
| | difference of materials before and after heating or cooling |
| | (unit: ° C.). (get the specific heat capacity of silicone |
| | rubber by searching c = 1.27 J/g*° C.) |
| Substitute the data into formula 1 to | Q = 1.27 (J/g*° C.)*56 g*18° C. = 1280.16 J |
| calculate the amount of heat absorbed | |
| by the funnel-shaped cover from 22° C. | |
| to 40° C. | |
| According to a calculation formula | formula 2: W = Q/t, W represents work done by the |
| | energized heating element (unit: w); t represents time |
| | necessary to heat the funnel-shaped cover to a preset |
| | temperature (unit: s). |
| Substitute the data into formula 2; the | W = 1280.16/30 = 42.67 W |
| work required for a heating wire to | |
| release heat | |
| The heating wire does not work | During suctioning milk, the heating wire has total power |
| continuously. Apart from heating to | of = 42.67 W*0.58 = 24.75 Wh; therefore, it is |
| 40° C. in the first 2 minutes, it then stops | expected to use a lithium battery of 3.7 V/6700 mAh = |
| heating. After it cools down to 38° C., try | 24.79 Wh. |
| to heating again to 40° C. Assuming it | |
| takes 30 seconds to drop from 40° C. to | |
| 38° C. and 30 seconds to heat from 38° C. | |
| to 40° C., the total power-consuming | |
| heating time for the wire is 35 minutes, | |
| and in the remaining 35 minutes there is | |
| no consumption of electricity with heat | |
| retention. 35 min = 0.58 h | |
| If the heating efficiency is calculated | A battery of 24.79 + 24.79* (1-80%) = 29.75 Wh |
| at 80% | is actually required |
| The total required lithium battery | Because the breast pump operates for 70 minutes and |
| capacity is | requires 2.16 Wh, the total battery capacity needs to |
| | be at least 2.16 + 29.75 = 31.91 Wh = |
| | 3.7 V/8700 mAh |

Note:
The above calculations give theoretical values, and there may be some deviations in practice.

As Embodiment 3 provided by the present invention (not shown in the figures), the heating apparatus includes:
an induction coil mounted on the heating cover, and the induction coil being electrically connected to the power supply device by joining the contacts set on the heating cover and the contacts set on the casing 21 together, and
the metal induction heating element 3 embedded inside the funnel-shaped cover 12 . . .

The controller is used to control a current alternating fluctuation in a circuit inside a coil holder by matching a PWM waveform corresponding to the heating mode, and the induction coil is used to generate an alternating induced magnetic field synchronous with the current alternating fluctuation, and the metal induction heating element is used to synchronously produce eddy currents and heat in the alternating induced magnetic field, so as to control a rate at which the funnel-shaped cover 12 reaches a preset temperature.

As an embodiment provided by the present invention, a hot-compress switch and a touch display interface are arranged on the casing 21 of the principal unit 2. This touch display interface may be a LED display, a LCD display, or a LED light indicator, and is used to control the wearable hot compress breast pump to turn on and turn off the heating function, and to select a hot-compress temperature. In this embodiment, three milk-suctioning control buttons are further arranged on the casing 21, and used to control the wearable hot compress breast pump to turn on and turn off, switch modes, and raise or lower a position of settings.

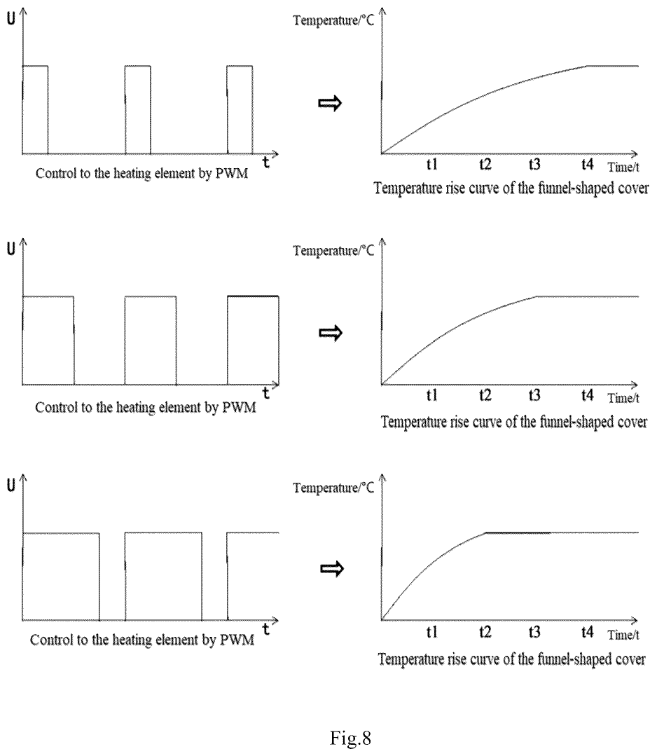

As shown in FIGS. 8-10, a method for controlling the wearable hot-compress breast pump provided by the present invention includes the following steps:
inputting a corresponding hot-compress control command into a control interface of a principal unit 2 on demands;
enabling a controller to receive the hot-compress control command and generate a corresponding current-controlling command;
enabling a power supply device to heat a heating apparatus in a corresponding heating mode according to the current-controlling command;
enabling a temperature sensor 6 to sense a temperature of the heating apparatus and feed the temperature back to the controller;
enabling the controller to judge whether the temperature of the heating apparatus has reached a preset threshold of the corresponding heating mode, if yes, controlling the power supply device to stop heating the heating apparatus; if not, controlling the power supply device to continue to heat the heating apparatus in a corresponding heating mode until the temperature of the heating apparatus reaches the preset threshold of the corresponding heating mode, then making the wearable hot-compress breast pump enter a heat retention mode.

In one example, the step of enabling a power supply device to heat a heating apparatus in a corresponding heating mode according to the current-controlling command, includes the following sub-steps.

As shown FIG. 8, enabling the controller to regulate heating power of a heating element 3 by matching a PWM waveform corresponding to the heating mode, so as to control a rate at which a funnel-shaped cover 12 reaches a preset temperature. By way of controlling the heating element 3 through the MCU's PWM waveform, it is possible to control a temperature rise rate of the funnel-shaped cover 12, preventing the funnel-shaped cover 12 from locally overheating due to a rapid temperature rise.

In other examples, the step of enabling a power supply device to heat a heating apparatus in a corresponding heating mode according to the current-controlling command, includes the following sub-steps.

Enabling the controller to control a current alternating fluctuation in a circuit inside a coil holder by matching a PWM waveform corresponding to the heating mode, then enabling an induction coil to generate an alternating induced magnetic field synchronous with the current alternating fluctuation, next enabling a metal induction heating element 3 to synchronously produce eddy currents and heat in the alternating induced magnetic field, so as to control a rate at which a funnel-shaped cover 12 reaches a preset temperature.

It is possible for a product according to the present invention to apply a hot compress massage on breasts while suctioning milk. In cases of using a wearable hot-compress breast pump in colder weather, it is possible effectively alleviate stimulation to skins of the breasts and chest caused by low temperatures. When lower temperatures stimulate the skins of the breasts and chest, the blood vessels in the breasts constrict, severely affecting users' experience and efficiency of suctioning milk. The product according to the present invention can turn off the hot-compress function in hot weather without affecting normal use. In frigid weather, it is possible to first turn on the hot-compress function to preheat the funnel-shaped cover 12 in contact with breasts, after reaching a preset temperature, wear the breast pump to suction milk; in this way, it is possible to set a preheated temperature on its own according to users' personal preference. When air temperature is low, the hot-compress function can be activated. The funnel-shaped cover in contact with the breasts will generate a mild temperature slightly higher than the body temperature, allowing users to feel cozy, stay comfortable, and enhance use experience. In addition, the mild temperature can also have an effect of applying a hot compress massage on the breasts, making it possible to effectively decongest a mammary gland, accelerate milk flow, increase milk production, and improve breastfeeding efficiency. In addition, it is possible to effectively relieve discomfort during breastfeeding and has a noticeable effect on alleviating breast soreness and lumps.

The above description is merely preferred embodiments of the present invention and are not intended to pose any limitations on the present invention. Any modifications, equivalent substitutions, and improvements made within the essence and principle of the present invention should fall within the protection scope of the present invention.

1. A wearable hot-compress breast pump, comprising:
a milk suctioning piece, which has one end serving as a milk outlet tube and the other end serving as a funnel-shaped cover, which communicates with the milk outlet tube, and an outer diameter of which gradually increases in a direction away from the milk outlet tube;
a principal unit, which has a casing, on one end of which a milk-storing chamber is disposed, and on the other end of which a funnel-shaped chamber that matches the shape of the funnel-shaped cover is disposed, wherein the milk-storing chamber has a milk inlet tube communicating with the funnel-shaped chamber,
the milk suctioning piece is stuck into the interior of the inlet tube through the outlet tube, so as to connect the principal unit;
a heating apparatus, which is disposed on one side of the funnel-shaped cover facing the milk outlet tube and connected with the funnel-shaped cover;
a power supply device and a controller, which are arranged inside the casing and electrically connected to the heating apparatus; and
a temperature sensor, which is embedded inside the funnel-shaped cover and electrically connected to the power supply device.

2. The wearable hot-compress breast pump according to claim 1, wherein the heating apparatus includes at least one heating element, which is embedded in a side surface of the funnel-shaped cover facing the milk outlet tube and the heating element extends around a surface contour of the funnel-shaped cover in a spiral manner.

3. The wearable hot-compress breast pump according to claim 2, wherein the heating apparatus further includes a heating cover, which is in the shape of a funnel that matches the shape of the funnel-shaped cover, and the heating element is arranged on one side of the heating cover facing the funnel-shaped cover, and the heating cover is provided with a mounting hole that matches the shape of the milk outlet tube, and the heating cover encases an outside of the milk outlet tube through the mounting hole and fits onto one side of the funnel-shaped cover facing the milk outlet tube, and encloses the heating element between the heating cover and the funnel-shaped cover.

4. The wearable hot-compress breast pump according to claim 3, wherein four contacts are set on one side of the funnel-shaped cover facing the milk outlet tube, and correspondingly connected to positive and negative terminals of the heating element and positive and negative terminals of the temperature sensor respectively; and four contacts electrically connected to the power supply device are set on the other end of the casing, and correspondingly connected to the four contacts set on the funnel-shaped cover respectively.

5. The wearable hot-compress breast pump according to claim 4, wherein the four contacts of the funnel-shaped cover and the four contacts of the casing are correspondingly connected with each other as a whole by means of a waterproof coating, and a magnetic connection is formed through pair magnets between the four contacts of the funnel-shaped cover and the four contacts of the casing, which have been connected with each other as a whole.

6. The wearable hot-compress breast pump according to claim 3, wherein the heating apparatus includes:
an induction coil mounted on the heating cover, the induction coil being electrically connected to the power supply device by joining the contacts set on the heating cover and the contacts set on the casing together; and
a metal induction heating element embedded inside the funnel-shaped cover.

7. The wearable hot-compress breast pump according to claim 2, wherein the heating element is any one of a resistive heating sheet, a silicone heating sheet, a mica heating sheet, a MCH heating sheet, a ceramic heating sheet, a silicon-conductive heating sheet, a flexible carbon film underfloor heating sheet, a PET electrothermal film polyester heating sheet, an aluminum foil heating sheet, a cushion heating sheet, a new-type surface heating sheet and a new-type thin-film heating sheet, and the heating element is in the form of strips, sheets, wires, or planes.

8. A method for controlling the wearable hot-compress breast pump according to claim 1, comprising the steps of inputting a corresponding hot-compress control command into a control interface of the principal unit on demands;
enabling the controller to receive the hot-compress control command and generate a corresponding current-controlling command;
enabling the power supply device to heat the heating apparatus in a corresponding heating mode according to the current-controlling command;
enabling the temperature sensor to sense a temperature of the heating apparatus and feed the temperature back to the controller; and
enabling the controller to judge whether the temperature of the heating apparatus has reached a preset threshold of the corresponding heating mode, if yes, controlling the power supply device to stop heating the heating apparatus; if not, controlling the power supply device to continue to heat the heating apparatus in a corresponding heating mode until the temperature of the heating apparatus reaches the preset threshold of the corresponding heating mode, then making the wearable hot-compress breast pump enter a heat retention mode.

9. The method according to claim 8, wherein the step of enabling the power supply device to heat the heating apparatus in a corresponding heating mode according to the current-controlling command, includes the sub-step of
enabling the controller to regulate heating power of the heating element by matching a PWM waveform corresponding to the heating mode, so as to control a rate at which the funnel-shaped cover reaches a preset temperature.

10. The method according to claim 8, wherein the step of enabling the power supply device to heat the heating apparatus in a corresponding heating mode according to the current-controlling command, includes the sub-steps of
enabling the controller to control a current alternating fluctuation in a circuit inside a coil holder by matching a PWM waveform corresponding to the heating mode, then enabling an induction coil to generate an alternating induced magnetic field synchronous with the current alternating fluctuation, next enabling a metal induction heating element to synchronously produce eddy currents and heat in the alternating induced magnetic field, so as to control a rate at which the funnel-shaped cover reaches a preset temperature.
